(19) United States
(12) Patent Application Publication
(10) Pub. No.: US 2014/0314787 A1
(43) Pub. Date: Oct. 23, 2014

(54) Treatment for neurodegenerative diseases

(71) Applicant: NOVARTIS FORSCHUNGSSTIFTUNG, ZWEIGNIEDERLASSUNG, FRIEDRICH MIESCHER INSTITUTE, (unknown)

(72) Inventors: Pico Caroni, Muttenz (CH); Francesco Roselli, Bari (IT)

(21) Appl. No.: 14/356,627

(22) PCT Filed: Nov. 7, 2012
(86) PCT No.: PCT/EP2012/072075

§ 371 Date: May 7, 2014

(30) Foreign Application Priority Data
Nov. 8, 2011 (EP) 11188250.2

(51) Int. Cl. C12N 15/113; C12Q 1/68; A61K 31/517; A61K 31/357; A61K 31/4178; C07K 16/40

(52) U.S. Cl. CPC C12N 15/1137; A61K 31/4178; C07K 16/40; A61K 31/517; A61K 31/357; C12Q 1/6883
USPC 424/146.1; 514/392; 514/44 A; 424/158.1; 514/266.4; 514/466; 506/9; 435/6.11; 435/6.12

(57) ABSTRACT

The present application provides a method for treating a neurodegenerative disease in a subject characterised in that the endoplasmic reticulum protein retrotranslocation machinery and/or VCP is modulated by administering to said subject a therapeutically effective amount of a modulator of said endoplasmic reticulum protein retrotranslocation machinery and/or of VCP.

### FIELD OF THE INVENTION

The present invention provides a method for the treatment of neurodegenerative diseases.

### BACKGROUND OF THE INVENTION

In subjects having neurodegenerative disease neurons of the brain and spinal cord are lost. Examples of neurodegenerative diseases include Alexander disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease (Spinocerebellar ataxia type 3), Multiple sclerosis, Multiple System Atrophy, Neuroborreliosis, Parkinson disease, Pelizaeus-Merzbacher Disease, Pick's disease, Primary lateral sclerosis, Prion diseases, Refsum's disease, Sandhoff disease, Schilder's disease, Sub-Acute Combined Degeneration of the Cord Secondary to Pernicious Anaemia, Schizophrenia, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tabes dorsalis and Charcot-Marie-Tooth disease.

Amyotrophic lateral sclerosis (ALS) is a fatal neurodegenerative disease that affects selectively motoneurons in the central nervous system. Most ALS patients die within five years of onset, and the mechanisms of the onset of the disease, as well as of its progression are poorly understood. Some of the present inventors have previously examined two mouse models of motoneuron (MN) disease (SOD1(G93A), SOD1(G85R)), and found that axons of fast-fatiguable (FF) MNs are affected synchronously, long before symptoms; fast-fatigue-resistant (FR) MN axons are affected at the onset of symptoms, and axons of slow (S) MNs are resistant (Pun et al., 2006, Nature Neuroscience, 9(3):408-419).

One of the main aspects in understanding the initiation and progression of a neurodegenerative disease such as in ALS is to elucidate mechanisms that underlie or predispose a particular neuron (in this case a MN) to selective vulnerability. In order to do this, new methods of early diagnostics are needed, that would allow to test preventive treatments.

There is hence a need in the art for such early diagnostics and treatment for neurodegeneration.

### SUMMARY OF THE INVENTION

The present inventors invented a new approach to detect very early the onset of neurodegenerative diseases, i.e. before the occurrence of any clinical symptom. Using this approach, the present inventors have surprisingly been able to identify a new therapy for neurodegenerative diseases.

The present inventors therefore provide a method for treating a neurodegenerative disease in a subject characterised in that the endoplasmic reticulum protein retrotranslocation machinery is modulated by administering to said subject a therapeutically effective amount of a modulator of said endoplasmic reticulum protein retrotranslocation machinery. In an embodiment of the invention, the modulator is a modulator of the AAA ATPase Valosin containing protein (VCP), for instance a VCP inhibitor. Said VCP inhibitor can be selected from the group consisting of Eeyarestatin-I, DBEQ and Syk-inhibitor III, and chemical derivatives thereof. The mode of action of the VCP modulator can be either through modulation of the endoplasmic reticulum protein retrotranslocation machinery or through the modulation of one of the pathways involving VCP.

In an embodiment of the invention, the neurodegenerative disease is Amyotrophic Lateral Sclerosis (ALS).

In a further embodiment of the invention, the modulator of the endoplasmic reticulum protein retrotranslocation machinery decreases or silences the expression of one or more component of the endoplasmic reticulum protein retrotranslocation machinery. The component of the endoplasmic reticulum protein retrotranslocation machinery can be the AAA ATPase Valosin containing protein (VCP). The modulator can be a siRNA.

In an alternative embodiment, the modulator of the endoplasmic reticulum protein retrotranslocation machinery can an antibody specifically binding to a component of the endoplasmic reticulum protein retrotranslocation machinery, for instance to the AAA ATPase Valosin containing protein (VCP). The antibody can be a monoclonal antibody.

Yet a further embodiment of the invention is a method of diagnosing a neurodegenerative disease comprising the step of assessing the expression of the gene coding for the AAA ATPase Valosin containing protein (VCP), or the level of the product coded by said gene, wherein an increase in the level of expression of said gene or in the level of said gene product, as compared to the levels measured in a normal population, is indicative of a possible neurodegenerative disease.

### DESCRIPTION OF THE FIGURES

FIG. 1: Climb test device

FIG. 2: Climb test detects early, gene-dosage dependent changes in the motor performance of mSOD1 trangenic mice.

FIG. 3: Eer-I delays motor disability in FSOD mice.

FIG. 4: Eer-I markedly improve FSOD mice performance in the climb test.

FIG. 5: Eer-I delays body weight loss.

FIG. 6: Eer-I prolongs FSOD mice survival.

FIG. 7: Eer-I treatment quickly restores performance of FSOD mice in the climb test.

FIG. 9: The ATP-site competitive VCP inhibitor DBEQ improves motor performance in FSOD mice.

FIG. 10: The syk-inhibitor III (known to inhibit also VCP) improves motor performance in FSOD mice.

FIG. 11 Eer-I reverses VCP upregulation in FSOD mice

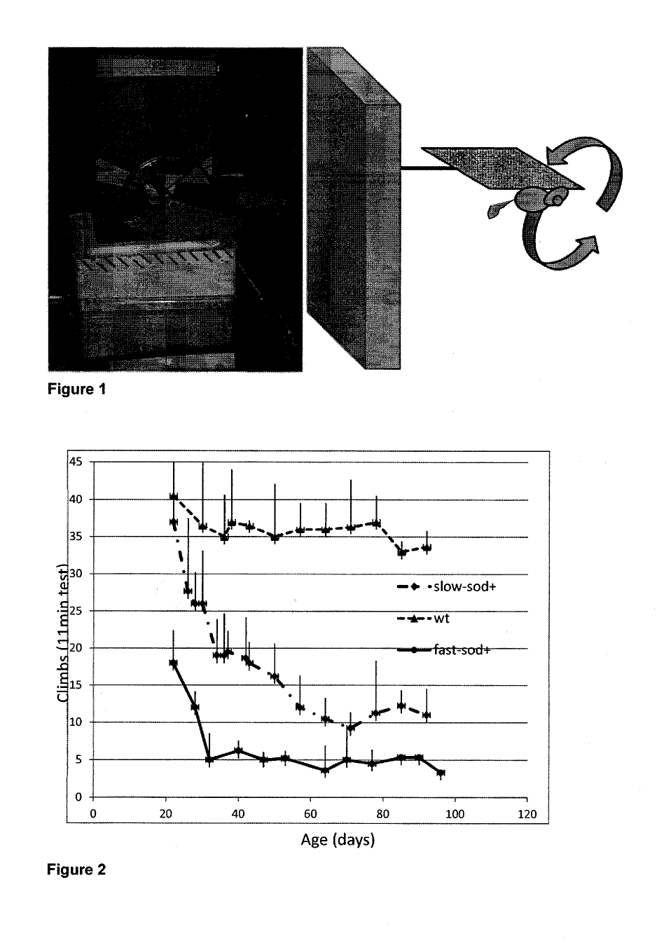

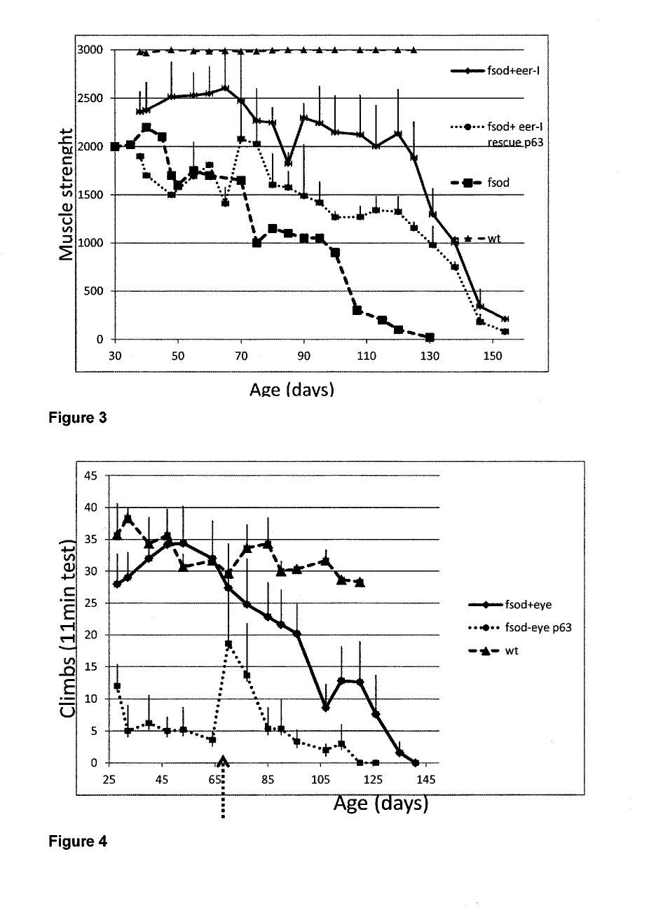

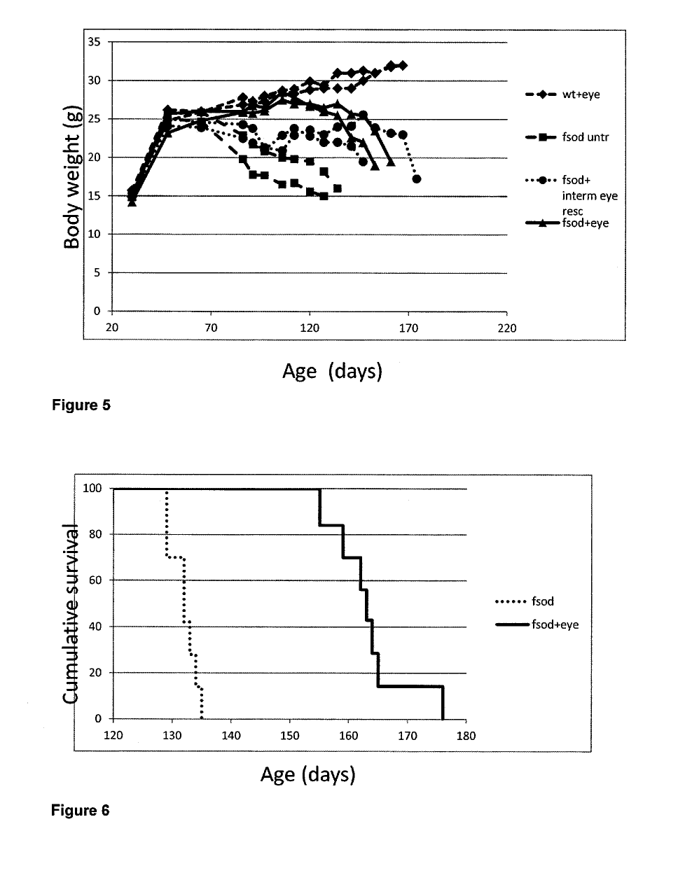

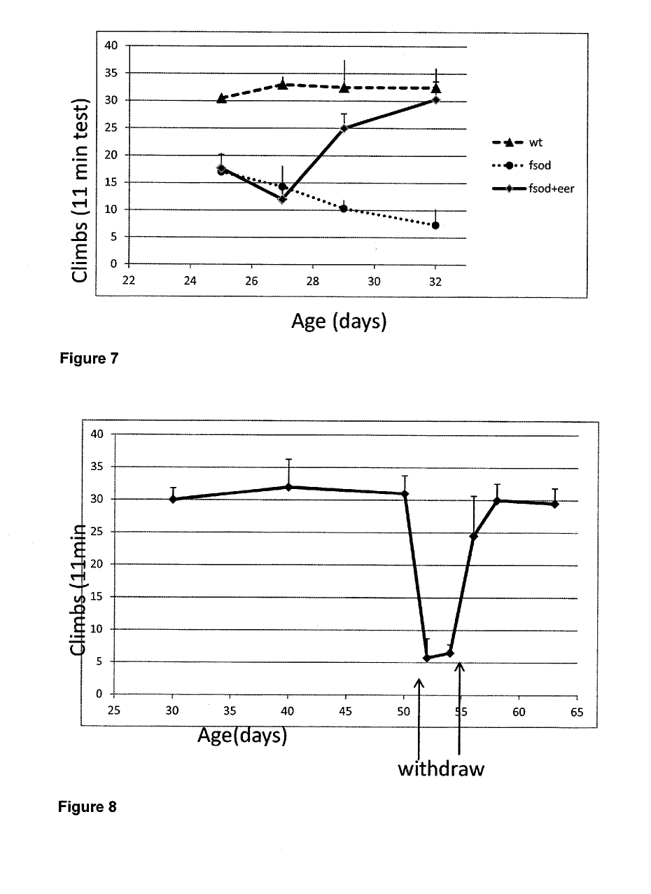

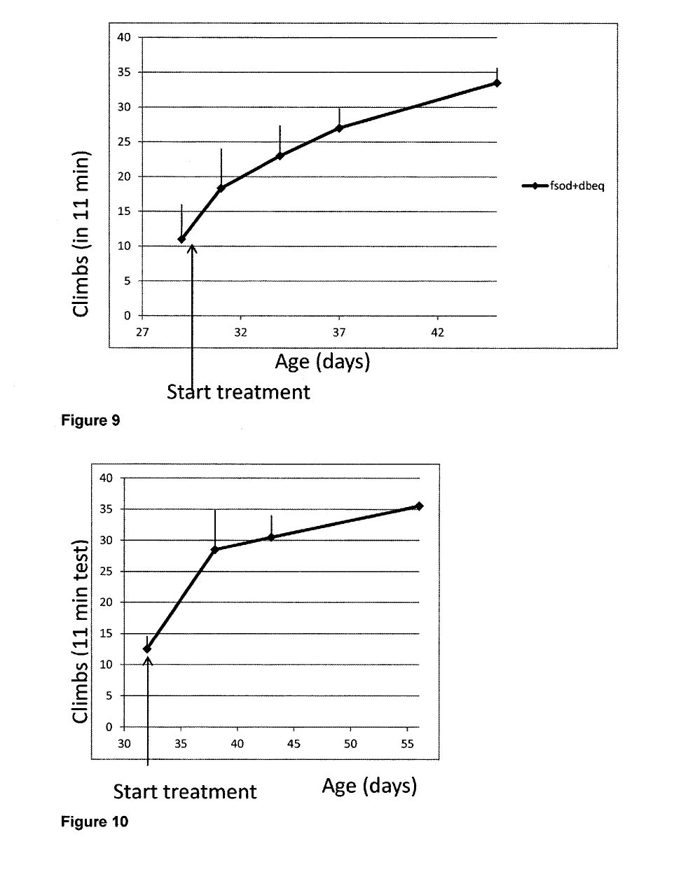

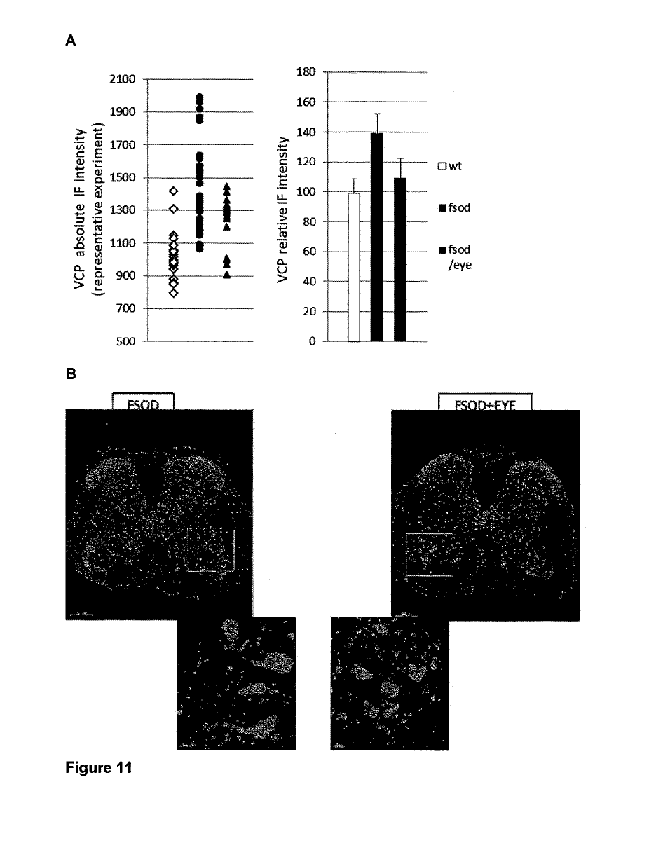

### DETAILED DESCRIPTION OF THE INVENTION

The present inventors invented a new approach to detect very early the onset of neurodegenerative diseases, i.e. before the occurrence of any clinical symptom. Using this approach, the present inventors have surprisingly been able to identify a new therapy for neurodegenerative diseases.

The present inventors therefore provide a method for treating a neurodegenerative disease in a subject characterised in that the endoplasmic reticulum protein retrotranslocation machinery is modulated by administering to said subject a therapeutically effective amount of a modulator of said endoplasmic reticulum protein retrotranslocation machinery. In an embodiment of the invention, the modulator is a modulator of the AAA ATPase Valosin containing protein (VCP), for instance a VCP inhibitor. Said VCP inhibitor can be selected from the group consisting of Eeyarestatin-I, DBEQ and Syk-inhibitor III, and chemical derivatives thereof. The mode of action of the VCP modulator can be either through modulation of the endoplasmic reticulum protein retrotranslocation machinery or through the modulation of one of the pathways involving VCP.

In an embodiment of the invention, the neurodegenerative disease is Amyotrophic Lateral Sclerosis (ALS).

In a further embodiment of the invention, the modulator of the endoplasmic reticulum protein retrotranslocation machinery decreases or silences the expression of one or more component of the endoplasmic reticulum protein retrotranslocation machinery. The component of the endoplasmic reticulum protein retrotranslocation machinery can be the AAA ATPase Valosin containing protein (VCP). The modulator can be a siRNA.

In an alternative embodiment, the modulator of the endoplasmic reticulum protein retrotranslocation machinery can an antibody specifically binding to a component of the endoplasmic reticulum protein retrotranslocation machinery, for instance to the AAA ATPase Valosin containing protein (VCP). The antibody can be a monoclonal antibody.

Yet a further embodiment of the invention is a method of diagnosing a neurodegenerative disease comprising the step of assessing the expression of the gene coding for the AAA ATPase Valosin containing protein (VCP), or the level of the product coded by said gene, wherein an increase in the level of expression of said gene or in the level of said gene product, as compared to the levels measured in a normal population, is indicative of a possible neurodegenerative disease.

The term “neurodegenerative disease” refers to a condition in which cells of the brain and spinal cord are lost. Examples of neurodegenerative diseases include, but are not limited to, Alexander disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease (Spinocerebellar ataxia type 3), Multiple sclerosis, Multiple System Atrophy, Neuroborreliosis, Parkinson disease, Pelizaeus-Merzbacher Disease, Pick's disease, Primary lateral sclerosis, Prion diseases, Refsum's disease, Sandhoff disease, Schilder's disease, Sub-Acute Combined Degeneration of the Cord Secondary to Pernicious Anaemia, Schizophrenia, Spielmeyer-Vogt-Sjogren-Batten disease (also known as Batten disease), Spinocerebellar ataxia (multiple types with varying characteristics), Spinal muscular atrophy, Steele-Richardson-Olszewski disease and Tabes dorsalis.

The term “motoneuron” or “motor neuron” applies to neurons located in the central nervous system (CNS) that project their axons outside the CNS and directly or indirectly control muscles. Motor neuron is also synonymous with efferent neuron. According to their targets, motoneurons are classified into three broad categories: “Somatic motoneurons”, which directly innervate skeletal muscles, involved in locomotion (such as muscles of the limbs, abdominal, and intercostal muscles), “Special visceral motoneurons”, also called “branchial motoneurons”, which directly innervate branchial muscles (that motorize the gills in fish and the face and neck in land vertebrates) and “General visceral motoneurons”, also termed “visceral motoneurons”, which indirectly innervate smooth muscles of the viscera (e.g. the heart, and the muscles of the arteries). Visceral motoneurons synapse onto neurons located in ganglia of the autonomic nervous system (sympathetic and parasympathetic), located in the peripheral nervous system (PNS), which themselves directly innervate visceral muscles (and also some gland cells). All motoneurons are cholinergic, i.e. they release the neurotransmitter acetylcholine. Parasympathetic ganglionic neurons are also cholinergic, whereas most sympathetic ganglionic neurons are noradrenergic, releasing the neurotransmitter noradrenaline. Somatic motoneurons are further subdivided into two types: alpha efferent neurons and gamma efferent neurons. “Alpha motoneurons” innervate extrafusal muscle fibers (also termed muscle fibers) located throughout the muscle. “Gamma motoneurons” innervate intrafusal muscle fibers found within the muscle spindle. In addition to voluntary skeletal muscle contraction, alpha motoneurons also contribute to muscle tone. Gamma motoneurons regulate the sensitivity of the spindle to muscle stretching.

Furthermore, alpha motoneurons can be further classified into the functional subtypes: fast-fatigable (FF), fast fatigue-resistant (FR) and slow (S) motoneurons, which show distinct excitability and recruitment properties and establish motor units (consisting of one motoneuron and all the muscle fibers it innervates) with markedly distinct fatigue and force properties (Burke, R. E. Physiology of motor units. in Myology (eds. Engel, A. G. & Franzini-Armstrong, C.) 464-484 (McGraw-Hill, New York, 1994)).

The term “motor neuron disease” or “motoneuron disease” comprises a group of severe disorders of the nervous system characterized by progressive degeneration of motor neurons (neurons are the basic nerve cells that combine to form nerves). Motor neurons control the behavior of muscles. Motor neuron diseases may affect the upper motor neurons, nerves that lead from the brain to the medulla (a part of the brain stem) or to the spinal cord, or the lower motor neurons, nerves that lead from the spinal cord to the muscles of the body, or both. Spasms and exaggerated reflexes indicate damage to the upper motor neurons. A progressive wasting (atrophy) and weakness of muscles that have lost their nerve supply indicate damage to the lower motor neurons. Examples of motor neuron diseases include, but are not limited to, Progressive Bulbar Palsy, Amyotrophic Lateral Sclerosis (ALS), Spinal Muscular Atrophy, Kugelberg-Welander Syndrome, Lou Gehrig's Disease, Duchenne's Paralysis, Werdnig-Hoffmann Disease, Juvenile Spinal Muscular Atrophy, Benign Focal Amyotrophy and Infantile Spinal Muscular Atrophy.

As used herein, the term “population” may be any group of at least two individuals. A population may include, e.g., but is not limited to, a reference population, a population group, a family population, a clinical population, and a same sex population.

As used herein, the term “polymorphism” means any sequence variant present at a frequency of >1% in a population. The sequence variant may be present at a frequency significantly greater than 1% such as 5% or 10% or more. Also, the term may be used to refer to the sequence variation observed in an individual at a polymorphic site. Polymorphisms include nucleotide substitutions, insertions, deletions and microsatellites and may, but need not, result in detectable differences in gene expression or protein function.

As used herein, the term “polynucleotide” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified e.g. for stability or for other reasons.

As used herein, the term “polypeptide” means any polypeptide comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well-known in the art. Such modifications are well described in basic texts and in more detailed monographs, as well as in a voluminous research literature.

As used herein, the term “reference standard population” means a population characterized by one or more biological characteristics, e.g., drug responsiveness, genotype, haplotype, phenotype, etc.

As used herein, the term “subject” means that preferably the subject is a mammal, such as a human, but can also be an animal, including but not limited to, domestic animals (e.g., dogs, cats and the like), farm animals (e.g., cows, sheep, pigs, horses and the like) and laboratory animals (e.g., monkeys such as cynmologous monkeys, rats, mice, guinea pigs and the like).

As used herein, a “test sample” means a biological sample obtained from a subject of interest. For example, a test sample can be a biological fluid (e.g., serum), cell sample, or tissue, or isolated nucleic acid or polypeptide derived therefrom.

As used herein, the expression “body fluid” is a biological fluid selected from a group comprising blood, bile, blood plasma, serum, aqueous humor, amniotic fluid, cerebrospinal fluid, sebum, intestinal juice, semen, sputum, sweat and urine.

As used herein, the term “dysregulation” means a change that is larger or equal to 1.2 fold and statistically significant (p<0.05, Student's t-test) from the control. For example, a 1.5, 2, 2.5, 3, 3.5, 4, 4.5, or 5 fold change.

As used herein, the term “statistically significant” means a p value <0.05 as compared to the control using the Student's t-test.

As used herein, “imminent” means that the onset of an event, e.g. neurodegeneration, will happen at the latest within five years. It is however to be understood that this term is relative and depends, for example, of the nature of the subject. In certain subjects, e.g. mice, this timeline will be much shorter. Alternatively, imminent neurodegenerative events can already have started, without however showing a phenotype yet.

The phrase “hybridising specifically to” as used herein refers to the binding, duplexing, or hybridising of an oligonucleotide probe preferentially to a particular target nucleotide sequence under stringent conditions when that sequence is present in a complex mixture (such as total cellular DNA or RNA).

Preferably a probe may bind, duplex or hybridise only to the particular target molecule.

The term “stringent conditions” refers to conditions under which a probe will hybridise to its target subsequence, but minimally to other sequences. Preferably a probe may hybridise to no sequences other than its target under stringent conditions. Stringent conditions are sequence-dependent and will be different in different circumstances. Longer sequences hybridise specifically at higher temperatures.

In general, stringent conditions may be selected to be about 5° C. lower than the thermal melting point (Tm) for the specific sequence at a defined ionic strength and pH. The Tm is the temperature (under defined ionic strength, pH, and nucleic acid concentration) at which 50% of the oligonucleotide probes complementary to a target nucleic acid hybridise to the target nucleic acid at equilibrium. As the target nucleic acids will generally be present in excess, at Tm, 50% of the probes are occupied at equilibrium. By way of example, stringent conditions will be those in which the salt concentration is at least about 0.01 to 1.0 M Na⁺ ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

Oligonucleotide probes may be used to detect complementary nucleic acid sequences (i.e., nucleic acid targets) in a suitable representative sample. Such complementary binding forms the basis of most techniques in which oligonucleotides may be used to detect, and thereby allow comparison of, expression of particular genes. Preferred technologies permit the parallel quantitation of the expression of multiple genes and include technologies where amplification and quantitation of species are coupled in real-time, such as the quantitative reverse transcription PCR technologies and technologies where quantitation of amplified species occurs subsequent to amplification, such as array technologies.

Array technologies involve the hybridisation of samples, representative of gene expression within the subject or control sample, with a plurality of oligonucleotide probes wherein each probe preferentially hybridises to a disclosed gene or genes. Array technologies provide for the unique identification of specific oligonucleotide sequences, for example by their physical position (e.g., a grid in a two-dimensional array as commercially provided by Affymetrix Inc.) or by association with another feature (e.g. labelled beads as commercially provided by Illumina Inc or Luminex Inc). Oligonuleotide arrays may be synthesised in situ (e.g by light directed synthesis as commercially provided by Affymetrix Inc) or pre-formed and spotted by contact or ink-jet technology (as commercially provided by Agilent or Applied Biosystems). It will be apparent to those skilled in the art that whole or partial cDNA sequences may also serve as probes for array technology (as commercially provided by Clontech).

Oligonucleotide probes may be used in blotting techniques, such as Southern blotting or northern blotting, to detect and compare gene expression (for example by means of cDNA or mRNA target molecules representative of gene expression). Techniques and reagents suitable for use in Southern or northern blotting techniques will be well known to those of skill in the art. Briefly, samples comprising DNA (in the case of Southern blotting) or RNA (in the case of northern blotting) target molecules are separated according to their ability to penetrate a gel of a material such as acrylamide or agarose. Penetration of the gel may be driven by capillary action or by the activity of an electrical field. Once separation of the target molecules has been achieved these molecules are transferred to a thin membrane (typically nylon or nitrocellulose) before being immobilized on the membrane (for example by baking or by ultraviolet radiation). Gene expression may then be detected and compared by hybridisation of oligonucleotide probes to the target molecules bound to the membrane.

In certain circumstances the use of traditional hybridisation protocols for comparing gene expression may prove problematic. For example blotting techniques may have difficulty distinguishing between two or more gene products of approximately the same molecular weight since such similarly sized products are difficult to separate using gels. Accordingly, in such circumstances it may be preferred to compare gene expression using alternative techniques, such as those described below.

Gene expression in a sample representing gene expression in a subject may be assessed with reference to global transcript levels within suitable nucleic acid samples by means of high-density oligonucleotide array technology. Such technologies make use of arrays in which oligonucleotide probes are tethered, for example by covalent attachment, to a solid support. These arrays of oligonucleotide probes immobilized on solid supports represent preferred components to be used in the methods and kits of the invention for the comparison of gene expression. Large numbers of such probes may be attached in this manner to provide arrays suitable for the comparison of expression of large numbers of genes selected from those listed above and in Table 2. Accordingly it will be recognised that such oligonucleotide arrays may be particularly preferred in embodiments of the methods of the invention where it is desired to compare expression of more than one gene of the invention.

Other suitable methodologies that may be used in the comparison of nucleic acid targets representative of gene expression include, but are not limited to, nucleic acid sequence based amplification (NASBA); or rolling circle DNA amplification (RCA).

The expression “axon-protecting factors” refers to factors protecting motoneurons from neurodegenerative diseases. The expression “axon-protecting factors” includes neutrophic factors. Neurotrophic factors have been suggested as potential therapeutic agents for motor neuron diseases (Thoenen et al., Exp. Neurology 124, 47-55, 1993). Indeed, embryonic motor neuron survival in culture is enhanced by members of the neurotrophin family such as brain derived neurotrophic factor (BDNF), neurotrophin-3 (NT-3), NT-4 (NT-4/5), cytokines such as ciliary neurotrophic factor (CNTF), leukaemia inhibitory factor (LIF) and cardiotrophin-1, glial cell line-derived neurotrophic factor (GDNF), insulin-like growth factor-1 (IGF-1) and members of the FGF family (review in Henderson, Neurotrophic factors as therapeutic agents in amyotrophic lateral sclerosis: potential and pitfalls. In Serratrice G. T. and Munsat T. L. eds. Pathogenesis and therapy of amyotrophic lateral sclerosis. Advances in Neurology, 68, pp. 235-240, 1995. Lippincott-Raven publishers, Philadelphia; Pennica et al., Cardiotrophin-1, a cytokine present in embryonic muscle, supports long-term survival of spinal motoneurons. Neuron, 17, 63-74, 1996). In vivo, a reduction of motoneuronal death occurring naturally during embryonic development was observed with CNTF (Oppenheim et al., Control of embryonic motoneuron survival in vivo by ciliary neurotrophic factor. Science, 251, 1616-1618, 1991), BDNF (Oppenheim et al., Brain-derived neurotrophic factor rescues developing avian motoneurons from cell death. Nature, 360, 755-757, 1992), GDNF (Oppenheim et al., Developing motor neurons rescued from programmed and axotomy-induced cell death by GDNF. Nature, 373, 344-346, 1995), and cardiotrophin-1 (Pennica et al., 1996). Protection from retrograde motor neuron death after acute peripheral nerve axotomy in neonate rodents was evidenced with several factors (Sendtner et al., Ciliary neurotroptuc factor prevents the degeneration of motor neurons after axotomy, Nature 345, 440-441, 1990, Sendtner et al, Ciliary neurotrophic factor prevents degeneration of motor neurons in mouse mutant progressive motor neuronopathy. Nature, 358, 502-504, 1992; Sendtner et al., Brain-derived neurotrophic prevents the death of motoneurons in newborn rats after nerve section. Nature, 360, 757-759, 1992; Vejsada et al., Quantitative comparison of the transient rescue effects of neurotrophic factors on axotomised motoneurons in vivo. Eur. J. Neurosci., 7, 108-115, 1995). Also, a protective effect of CNTF and/or BDNF was described in two murine models of inherited progressive motor degeneration (Sendtner et al., 1992; Mitsumoto et al., Arrest of motor neuron disease in wobbler mice cotreated with CNTF and BDNF. Science, 265, 1107-1110, 1994). The preferred neurotrophic factors are ciliary neurotrophic factor (CNTF), glial cell maturation factors (GMFa, b), GDNF, BDNF, NT-3, NT-5 and the like. The neurotrophic factor NT-3 is particularly preferred. The complete nucleotide sequence encoding NT-3 is disclosed in WO91/03569, the contents of which are incorporated herein by reference.

The term “epitopes,” as used herein, refers to portions of a polypeptide having antigenic or immunogenic activity in an animal, in some embodiments, a mammal,for instance in a human. In an embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immuno specifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic. Fragments which function as epitopes may be produced by any conventional means. (See, e.g., Houghten, Proc. Natl. Acad. Sci. USA 82:5131-5135 (1985), further described in U.S. Pat. No. 4,631,211).

As one of skill in the art will appreciate, and as discussed above, polypeptides comprising an immunogenic or antigenic epitope can be fused to other polypeptide sequences. For example, polypeptides may be fused with the constant domain of immunoglobulins (IgA, IgE, IgG, IgM), or portions thereof (CH1, CH2, CH3, or any combination thereof and portions thereof), or albumin (including but not limited to recombinant albumin (see, e.g., U.S. Pat. No. 5,876,969, issued Mar. 2, 1999, EP Patent 0 413 622, and U.S. Pat. No. 5,766,883, issued Jun. 16, 1998)), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988).

Enhanced delivery of an antigen across the epithelial barrier to the immune system has been demonstrated for antigens (e.g., insulin) conjugated to an FcRn binding partner such as IgG or Fc fragments (see, e. g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Blochem., 270:3958-3964 (1995). Nucleic acids encoding the above epitopes can also be recombined with a gene of interest as an epitope tag (e.g., the hemagglutinin (“HA”) tag or flag tag) to aid in detection and punification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein.

Extracts from cells infected with the recombinant vaccinia virus are loaded onto Ni²⁺ nitriloacetic acid-agarose column and histidine-tagged proteins can be selectively eluted with imidazole-containing buffers. Additional fusion proteins may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998).

Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. In addition, in the context of the present invention, the term “antibody” shall also encompass alternative molecules having the same function, e.g. aptamers and/or CDRs grafted onto alternative peptidic or non-peptidic frames. In some embodiments the antibodies are human antigen-binding antibody fragments and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. In some embodiments, the antibodies are human, murine (e.g., mouse and rat), donkey, ship rabbit, goat, guinea pig, camel, shark, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al. The antibodies of the present invention may be monospecific, bispecific, trispecific or of greater multi specificity.

Multispecific antibodies may be specific for different epitopes of a polypeptide or may be specific for both a polypeptide as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide which they recognize or specifically bind. The epitope(s) or polypeptide portion(s) may be specified as described herein, e.g., by N-terminal and C-terminal positions, by size in contiguous amino acid residues. Antibodies may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide are also included in the present invention. In specific embodiments, antibodies of the present invention cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%. less than 55%, and less than 50% identity (as calculated using methods known in the art and described herein) to a polypeptide are also included in the present invention.

Antibodies may also be described or specified in terms of their binding affinity to a polypeptide Antibodies may act as agonists or antagonists of the recognized polypeptides. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are antibodies which bind the ligand, thereby preventing receptor activation, but do not prevent the ligand from binding the receptor. The antibodies may be specified as agonists, antagonists or inverse agonists for biological activities comprising the specific biological activities of the peptides disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III(Pt2) :237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(I):14-20 (1996).

As discussed in more detail below, the antibodies may be used either alone or in combination with other compositions. The antibodies may further be recombinantly fused to a heterologous polypeptide at the N-or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396, 387. The antibodies as defined for the present invention include derivatives that are modified, i. e, by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen.

Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvurn. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CHI domain of the heavy chain.

For example, the antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al, Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. As described in these references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax. et al., BioTechniques 12(6):864-869 (1992); and Sawai et al., AJRI 34:26-34 (1995); and Better et al., Science 240:1041-1043 (1988).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, and/or improve, antigen binding. These framework substitutions are identified by methods well known in the art, e.g., by modelling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988).) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592, 106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences.

See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harboured by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immurnol. 13:65-93 (1995). For a detailed discussion of this technology for producing human antibodies and human monoclonal antibodies and protocols for producing such antibodies, see, e. g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598. In addition, companies such as Abgenix, Inc. (Freemont, Calif.) and Genpharm (San Jose, Calif.) can be engaged to provide human antibodies directed against a selected antigen using technology similar to that described above. Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)). Furthermore, antibodies can be utilized to generate anti-idiotype antibodies that “mimic” polypeptides using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization. and/or binding of a polypeptide to a ligand can be used to generate anti-idiotypes that “mimic” the polypeptide multimerization. and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide and/or to bind its ligands/receptors, and thereby block its biological activity. Polynucleotides encoding antibodies, comprising a nucleotide sequence encoding an antibody are also encompassed. These polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

The amino acid sequence of the heavy and/or light chain variable domains may be inspected to identify the sequences of the complementarity determining regions (CDRs) by methods that are well known in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and in some embodiments, human framework regions (see, e.g., Chothia et al., J. Mol. Biol. 278: 457-479 (1998) for a listing of human framework regions). In some embodiments, the polynucleotide generated by the combination of the framework regions and CDRs encodes an antibody that specifically binds a polypeptide. In some embodiments, as discussed supra, one or more amino acid substitutions may be made within the framework regions, and, in some embodiments, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polymicleotide are encompassed by the present description and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

The present invention encompasses antibodies recombinantly fused or chemically conjugated (including both covalently and non-covalently conjugations) to a polypeptide (or portion thereof, in some embodiments, at least 10, 20, 30, 40, 50, 60, 70, 80, 90 or 100 amino acids of the polypeptide) to generate fusion proteins. The fusion does not necessarily need to be direct, but may occur through linker sequences. The antibodies may be specific for antigens other than polypeptides (or portion thereof, in some embodiments, at least 10, 20, 30, 40, 50, 60, 70, 80, 90 or 100 amino acids of the polypeptide). Further, an antibody or fragment thereof may be conjugated to a therapeutic moiety, for instance to increase their therapeutical activity. The conjugates can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, B-interferon, nerve growth factor, platelet derived growth factor, tissue plasminogen activator, an apoptotic agent, e.g., TNF-alpha, TNF-beta, AIM I (See, International Publication No. WO 97/33899), AIM 11 (See, International Publication No. WO 97/34911), Fas Ligand (Takahashi et aL, Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 (“IL-I”), interleukin-2 (“IL-2”), interleukin-6 (“IL-6”), granulocyte macrophage colony stimulating factor (“GM-CSF”), granulocyte colony stimulating factor (“G-CSF”), or other growth factors. Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982). Alternatively, an antibody can be conjugated to a second antibody to form an antibody heteroconjugate as described by Segal in U.S. Pat. No. 4,676,980.

The present invention is also directed to antibody-based therapies which involve administering antibodies of the invention to an animal, in some embodiments, a mammal, for example a human, patient to treat neurodegenerative diseases. Therapeutic compounds include, but are not limited to, antibodies (including fragments, analogs and derivatives thereof as described herein) and nucleic acids encoding antibodies of the invention (including fragments, analogs and derivatives thereof and anti-idiotypic antibodies as described herein). Antibodies of the invention may be provided in pharmaceutically acceptable compositions as known in the art or as described herein.

The invention also provides methods for treating neurodegenerative diseases in a subject by inhibiting the endoplasmic reticulum protein retrotranslocation machinery by administration to the subject of an effective amount of an inhibitory compound or pharmaceutical composition comprising an inhibitory compound of the endoplasmic reticulum protein retrotranslocation machinery. In some embodiments, said inhibitory compound is a small molecule, an antibody or a siRNA. In an embodiment, the compound is substantially purified (e.g., substantially free from substances that limit its effect or produce undesired side-effects). The subject is in some embodiments, an animal, including but not limited to animals such as cows, pigs, horses, chickens, cats, dogs, etc., and is in some embodiments, a mammal, for example human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound, e.g., encapsulation in liposomes, microparticles, microcapsules, recombinant cells capable of expressing the compound, receptor-mediated endocytosis (see, e. g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 317-327; see generally ibid.) In yet another embodiment, the compound or composition can be delivered in a controlled release system. In one embodiment, a pump may be used (see Langer, supra; Sefton, CRC Crit. Ref, Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g. , Goodson, in Medical Applications of Controlled Release, supra, vol. 2, pp. 115-138 (1984)). Other controlled release systems are discussed in the review by Langer (Science 249:1527-1533 (1990)).

The present invention also provides pharmaceutical compositions for use in the treatment of neurodegenerative diseases by inhibiting the endoplasmic reticulum protein retrotranslocation machinery. Such compositions comprise a therapeutically effective amount of an inhibitory compound, and a pharmaceutically acceptable carrier. In a specific embodiment, the term “pharmaceutically acceptable” means approved by a regulatory agency of the Federal or a state government or listed in the U. S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, tale, sodium chloride, driied skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, in some embodiments, in purified form, together with a suitable amount of carrier so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lidocaine to ease pain at the site of the injection.

Generally, the ingredients are supplied either separately or mixed together in unit dosage form, for example, as a dry lyophilized powder or water free concentrate in a hermetically scaled container such as an ampoule or sachette indicating the quantity of active agent.

Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms.

Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. The amount of the compound which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances.

Effective doses may be extrapolated from dose-response curves derived from in vitro or animal model test systems. For antibodies, the dosage administered to a patient is typically 0.1 mg/kg to 100 mg/kg of the patient's body weight. In some embodiments, the dosage administered to a patient is between 0.1 mg/kg and 20 mg/kg of the patient's body weight, for example 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

Also encompassed is a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The antibodies as encompassed herein may also be chemically modified derivatives which may provide additional advantages such as increased solubility, stability and circulating time of the polypeptide, or decreased immunogenicity (see U.S. Pat. No. 4,179,337). The chemical moieties for derivatisation may be selected from water soluble polymers such as polyethylene glycol, ethylene glycol/propylene glycol copolymers, carboxymethylcellulose, dextran, polyvinyl alcohol and the like.

The antibodies may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100000 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,600, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa. As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999). The polyethylene glycol molecules (or other chemical moieties) should be attached to the protein with consideration of effects, on functional or antigenic domains of the protein. There are a number of attachment methods available to those skilled in the art, e.g., EP 0 401 384 (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group. As suggested above, polyethylene glycol may be attached to proteins via linkage to any of a number of amino acid residues. For example, polyethylene glycol can be linked to proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein. As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466.

“siRNA” or “small-interfering ribonucleic acid” according to the invention has the meanings known in the art, including the following aspects. The siRNA consists of two strands of ribonucleotides which hybridize along a complementary region under physiological conditions. The strands are normally separate. Because of the two strands have separate roles in a cell, one strand is called the “anti-sense” strand, also known as the “guide” sequence, and is used in the functioning RISC complex to guide it to the correct mRNA for cleavage. This use of “anti-sense”, because it relates to an RNA compound, is different from the antisense target DNA compounds referred to elsewhere in this specification. The other strand is known as the “anti-guide” sequence and because it contains the same sequence of nucleotides as the target sequence, it is also known as the sense strand. The strands may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

In some embodiments, the siRNA molecule is substantially identical with at least a region of the coding sequence of the target gene to enable down-regulation of the gene. In some embodiments, the degree of identity between the sequence of the siRNA molecule and the targeted region of the gene is at least 60% sequence identity, in some embodiments at least 75% sequence identity, for instance at least 85% identity, 90% identity, at least 95% identity, at least 97%, or at least 99% identity.

Calculation of percentage identities between different amino acid/polypeptide/nucleic acid sequences may be carried out as follows. A multiple alignment is first generated by the ClustalX program (pairwise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared.

Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof. A substantially similar nucleotide sequence will be encoded by a sequence which hybridizes to any of the nucleic acid sequences referred to herein or their complements under stringent conditions. By stringent conditions, we mean the nucleotide hybridises to filter-bound DNA or RNA in 6×sodium chloride/sodium citrate (SSC) at approximately 45° C. followed by at least one wash in 0.2×SSC/0.1% SDS at approximately 5-65° C. Alternatively, a substantially similar polypeptide may differ by at least 1, but less than 5, 10, 20, 50 or 100 amino acids from the peptide sequences according to the present invention Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequences which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine; large non-polar, hydrophobic amino acids include phenylalanine, tryptophan and tyrosine; the polar neutral amino acids include serine, threonine, cysteine, asparagine and glutamine; the positively charged (basic) amino acids include lysine, arginine and histidine; and the negatively charged (acidic) amino acids include aspartic acid and glutamic acid. The accurate alignment of protein or DNA sequences is a complex process, which has been investigated in detail by a number of researchers. Of particular importance is the trade-off between optimal matching of sequences and the introduction of gaps to obtain such a match. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantify the nature and likelihood of conservative substitutions and are used in multiple alignment algorithms, although other, equally applicable matrices will be known to those skilled in the art. The popular multiple alignment program ClustalW, and its windows version ClustalX (Thompson et al., 1994, Nucleic Acids Research, 22, 4673-4680; Thompson et al., 1997, Nucleic Acids Research, 24, 4876-4882) are efficient ways to generate multiple alignments of proteins and DNA. Frequently, automatically generated alignments require manual alignment, exploiting the trained user's knowledge of the protein family being studied, e.g., biological knowledge of key conserved sites. One such alignment editor programs is Align (http://www.gwdg. de/dhepper/download/; Hepperle, D., 2001: Multicolor Sequence Alignment Editor. Institute of Freshwater Ecology and Inland Fisheries, 16775 Stechlin, Germany), although others, such as JalView or Cinema are also suitable. Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein; http://evolution.gs. washington.edu/phylip.html) using the “Similarity Table” option as the model for amino acid substitution (P). For DNA/RNA, an identical option exists within the DNADIST program of PHYL1P.

The dsRNA molecules in accordance with the present invention comprise a double-stranded region which is substantially identical to a region of the mRNA of the target gene. A region with 100% identity to the corresponding sequence of the target gene is suitable. This state is referred to as “fully complementary”. However, the region may also contain one, two or three mismatches as compared to the corresponding region of the target gene, depending on the length of the region of the mRNA that is targeted, and as such may be not fully complementary. In an embodiment, the RNA molecules of the present invention specifically target one given gene. In order to only target the desired mRNA, the siRNA reagent may have 100% homology to the target mRNA and at least 2 mismatched nucleotides to all other genes present in the cell or organism. Methods to analyze and identify siRNAs with sufficient sequence identity in order to effectively inhibit expression of a specific target sequence are known in the art. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

The length of the region of the siRNA complementary to the target, in accordance with the present invention, may be from 10 to 100 nucleotides, 12 to 25 nucleotides, 14 to 22 nucleotides or 15, 16, 17 or 18 nucleotides. Where there are mismatches to the corresponding target region, the length of the complementary region is generally required to be somewhat longer. In an embodiment, the inhibitor is a siRNA molecule and comprises between approximately 5 bp and 50 bp, in some embodiments, between 10 by and 35 bp, or between 15 by and 30 bp, for instance between 18 by and 25 bp. In some embodiments, the siRNA molecule comprises more than 20 and less than 23 bp.

Because the siRNA may carry overhanging ends (which may or may not be complementary to the target), or additional nucleotides complementary to itself but not the target gene, the total length of each separate strand of siRNA may be 10 to 100 nucleotides, 15 to 49 nucleotides, 17 to 30 nucleotides or 19 to 25 nucleotides.

The phrase “each strand is 49 nucleotides or less” means the total number of consecutive nucleotides in the strand, including all modified or unmodified nucleotides, but not including any chemical moieties which may be added to the 3′ or 5′ end of the strand. Short chemical moieties inserted into the strand are not counted, but a chemical linker designed to join two separate strands is not considered to create consecutive nucleotides.

The phrase “a 1 to 6 nucleotide overhang on at least one of the 5′ end or 3′ end” refers to the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created.

The overhang length is measured by the number of overhanging nucleotides. The overhanging nucleotides can be either on the 5′ end or 3′ end of either strand.

The siRNA according to the present invention display a high in vivo stability and may be particularly suitable for oral delivery by including at least one modified nucleotide in at least one of the strands.

Thus the siRNA according to the present invention contains at least one modified or non-natural ribonucleotide. A lengthy description of many known chemical modifications are set out in published PCT patent application WO 200370918. Suitable modifications for delivery include chemical modifications can be selected from among: a) a 3′ cap; b) a 5′ cap,c) a modified internucleoside linkage; or d) a modified sugar or base moiety.

Suitable modifications include, but are not limited to modifications to the sugar moiety (i.e. the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides) with for instance phosphorothioates, chiral phosphorothioates or phosphorodithioates.

End modifications sometimes referred to herein as 3′ caps or 5′ caps may be of significance. Caps may consist of simply adding additional nucleotides, such as “T-T” which has been found to confer stability on a siRNA. Caps may consist of more complex chemistries which are known to those skilled in the art.

Design of a suitable siRNA molecule is a complicated process, and involves very carefully analysing the sequence of the target mRNA molecule. On exemplary method for the design of siRNA is illustrated in WO2005/059132. Then, using considerable inventive endeavour, the inventors have to choose a defined sequence of siRNA which has a certain composition of nucleotide bases, which would have the required affinity and also stability to cause the RNA interference.

The siRNA molecule may be either synthesised de novo, or produced by a micro-organism. For example, the siRNA molecule may be produced by bacteria, for example, E. coli. Methods for the synthesis of siRNA, including siRNA containing at least one modified or non-natural ribonucleotides are well known and readily available to those of skill in the art. For example, a variety of synthetic chemistries are set out in published PCT patent applications WO2005021749 and WO200370918.

The reaction may be carried out in solution or, in some embodiments, on solid phase or by using polymer supported reagents, followed by combining the synthesized RNA strands under conditions, wherein a siRNA molecule is formed, which is capable of mediating RNAi.

It should be appreciated that siNAs (small interfering nucleic acids) may comprise uracil (siRNA) or thyrimidine (siDNA). Accordingly the nucleotides U and T, as referred to above, may be interchanged. However it is preferred that siRNA is used.

Gene-silencing molecules, i.e. inhibitors, used according to the invention are in some embodiments, nucleic acids (e.g. siRNA or antisense or ribozymes). Such molecules may (but not necessarily) be ones, which become incorporated in the DNA of cells of the subject being treated. Undifferentiated cells may be stably transformed with the gene-silencing molecule leading to the production of genetically modified daughter cells (in which case regulation of expression in the subject may be required, e.g. with specific transcription factors, or gene activators).

The gene-silencing molecule may be either synthesised de novo, and introduced in sufficient amounts to induce gene-silencing (e.g. by RNA interference) in the target cell. Alternatively, the molecule may be produced by a micro-organism, for example, E. coli, and then introduced in sufficient amounts to induce gene silencing in the target cell.

The molecule may be produced by a vector harbouring a nucleic acid that encodes the gene-silencing sequence. The vector may comprise elements capable of controlling and/or enhancing expression of the nucleic acid. The vector may be a recombinant vector. The vector may for example comprise plasmid, cosmid, phage, or virus DNA. In addition to, or instead of using the vector to synthesise the gene-silencing molecule, the vector may be used as a delivery system for transforming a target cell with the gene silencing sequence.

The recombinant vector may also include other functional elements. For instance, recombinant vectors can be designed such that the vector will autonomously replicate in the target cell. In this case, elements that induce nucleic acid replication may be required in the recombinant vector. Alternatively, the recombinant vector may be designed such that the vector and recombinant nucleic acid molecule integrates into the genome of a target cell. In this case nucleic acid sequences, which favour targeted integration (e.g. by homologous recombination) are desirable. Recombinant vectors may also have DNA coding for genes that may be used as selectable markers in the cloning process.

The recombinant vector may also comprise a promoter or regulator or enhancer to control expression of the nucleic acid as required. Tissue specific promoter/enhancer elements may be used to regulate expression of the nucleic acid in specific cell types, for example, endothelial cells. The promoter may be constitutive or inducible:

Alternatively, the gene silencing molecule may be administered to a target cell or tissue in a subject with or without it being incorporated in a vector. For instance, the molecule may be incorporated within a liposome or virus particle (e.g. a retrovirus, herpes virus, pox virus, vaccina virus, adenovirus, lentivirus and the like).

Alternatively a “naked” siRNA or antisense molecule may be inserted into a subject's cells by a suitable means e.g. direct endocytotic uptake.

The gene silencing molecule may also be transferred to the cells of a subject to be treated by either transfection, infection, microinjection, cell fusion, protoplast fusion or ballistic bombardment. For example, transfer may be by: ballistic transfection with coated gold particles; liposomes containing a siNA molecule; viral vectors comprising a gene silencing sequence or means of providing direct nucleic acid uptake (e.g. endocytosis) by application of the gene silencing molecule directly.

In an embodiment of the present invention siNA molecules may be delivered to a target cell (whether in a vector or “naked”) and may then rely upon the host cell to be replicated and thereby reach therapeutically effective levels. When this is the case the siNA is in some embodiments, incorporated in an expression cassette that will enable the siNA to be transcribed in the cell and then interfere with translation (by inducing destruction of the endogenous mRNA coding the targeted gene product). Inhibitors according to any embodiment of the present invention may be used in a monotherapy (e.g. use of siRNAs alone). However it will be appreciated that the inhibitors may be used as an adjunct, or in combination with other therapies.

The inhibitors of THE ENDOPLASMIC RETICULUM PROTEIN RETROTRANSLOCATION MACHINERY may be contained within compositions having a number of different forms depending, in particular on the manner in which the composition is to be used. Thus, for example, the composition may be in the form of a capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given, and in some embodiments, enables delivery of the inhibitor to the target site.

The inhibitors of the endoplasmic reticulum protein retrotranslocation machinerymay be used in a number of ways.

For instance, systemic administration may be required in which case the compound may be contained within a composition that may, for example, be administered by injection into the blood stream. Injections may be intravenous (bolus or infusion), subcutaneous, intramuscular or a direct injection into the target tissue (e.g. an intraventricular injection-when used in the brain). The inhibitors may also be administered by inhalation (e.g. intranasally) or even orally (if appropriate).

The inhibitors of the invention may also be incorporated within a slow or delayed release device. Such devices may, for example, be inserted in the body of the subject, and the molecule may be released over weeks or months. Such devices may be particularly advantageous when long term treatment with an inhibitor of the endoplasmic reticulum protein retrotranslocation machinery's required and which would normally require frequent administration (e.g. at least daily injection).

It will be appreciated that the amount of an inhibitor that is required is determined by its biological activity and bioavailability which in turn depends on the mode of administration, the physicochemical properties of the molecule employed and whether it is being used as a monotherapy or in a combined therapy. The frequency of administration will also be influenced by the above-mentioned factors and particularly the half-life of the inhibitor within the subject being treated.

Optimal dosages to be administered may be determined by those skilled in the art, and will vary with the particular inhibitor in use, the strength of the preparation, and the mode of administration.

Additional factors depending on the particular subject being treated will result in a need to adjust dosages, including subject age, weight, gender, diet, and time of administration.

When the inhibitor is a nucleic acid conventional molecular biology techniques (vector transfer, liposome transfer, ballistic bombardment etc) may be used to deliver the inhibitor to the target tissue.

Known procedures, such as those conventionally employed by the pharmaceutical industry (e.g. in vivo experimentation, clinical trials, etc.), may be used to establish specific formulations for use according to the invention and precise therapeutic regimes (such as daily doses of the gene silencing molecule and the frequency of administration).

Generally, a daily dose of between 0.01 μg/kg of body weight and 0.5 g/kg of body weight of an inhibitor of the endoplasmic reticulum protein retrotranslocation machinery may be used for the treatment of neurodegenerative diseases in the subject, depending upon which specific inhibitor is used. When the inhibitor is an siRNA molecule, the daily dose may be between 1 pg/kg of body weight and 100 mg/kg of body weight, in some embodiments, between approximately 10 pg/kg and 10 mg/kg, or between about 50 pg/kg and 1 mg/kg.

When the inhibitor (e.g. siNA) is delivered to a cell, daily doses may be given as a single administration (e.g. a single daily injection).

Various assays are known in the art to test dsRNA for its ability to mediate RNAi (see for instance Elbashir et al., Methods 26 (2002), 199-213). The effect of the dsRNA according to the present invention on gene expression will typically result in expression of the target gene being inhibited by at least 10%, 33%, 50%, 90%, 95% or 99% when compared to a cell not treated with the RNA molecules according to the present invention.

Similarly, various assays are well-known in the art to test antibodies for their ability to inhibit the biological activity of their specific targets. The effect of the use of an antibody according to the present invention will typically result in biological activity of their specific target being inhibited by at least 10%, 33%, 50%, 90%, 95% or 99% when compared to a control not treated with the antibody.

“AAA” or “AAA+” is an abbreviation for ATPases Associated with diverse cellular Activities. They share a common conserved module of approximately 230 amino acid residues. This is a large, functionally diverse protein family belonging to the AAA+ superfamily of ring-shaped P-loop NTPases, which exert their activity through the energy-dependent remodeling or translocation of macromolecules. These proteins are involved in a range of processes, including DNA replication, protein degradation, membrane fusion, microtubule severing, peroxisome biogenesis, signal transduction and the regulation of gene expression. The characteristic of AAA proteins is the coupling of chemical energy by ATPase, provided by ATP hydrolysis, to mechanical force exerted on some macromolecular substrate. This usually requires a conformational change in the AAA protein in question. AAA ATPases assemble into oligomeric assemblies (often hexamers) that form a ring-shaped structure with a central pore. These proteins produce a molecular motor that couples ATP binding and hydrolysis to changes in conformational states that can be propagated through the assembly in order to act upon a target substrate, either translocating or remodelling the substrate. Members of the AAA family are found in all organisms and they are essential for many cellular functions. One type of AAA Proteins are AAA Proteases, where the energy from ATP hydrolysis is used to translocate a Protein inside the Protease for degradation. AAA-type ATPases constitute a large family of enzymes. AAA proteins are characterised by the presence of 200-250 amino-acid ATP-binding domains that contain Walker A and Walker B motifs. AAA proteins themselves belong to the superfamily of P-loop NTPases. All AAA+ proteins have a mixed alpha/beta domain that binds and hydrolyzes nucleotide. Most AAA+ proteins have a second domain that comprises the AAA+ module: an all alpha-helical domain, often called the lid domain that is C-terminal of the alpha/beta domain. Most AAA+ proteins have additional domains that are used for oligomerization, substrate binding and/or regulation. These domains can lay N- or C-terminal to the AAA+ module. Some classes of AAA proteins have an N-terminal Non-ATPase domain which is followed by either one or two AAA domains (D1 and D2). In some proteins with two AAA domains, both are evolutionarily well conserved (like in Cdc48/p97). In others, either the D2 domain (like in Pex1 p and Pex6p) or the D1 domain (in Sec18p/NSF) is better conserved in evolution. The classical AAA family has been expanded by inclusion of a number of more distantly related cellular regulators and termed AAA+ family of ATPases. AAA+ proteins are involved in protein degradation, membrane fusion, DNA replication, microtubule dynamics, intracellular transport, flagellar and ciliary beating, disassembly of protein complexes and protein aggregates. The physiologically active form of these enzymes is often a homo-hexamer. The hexameric enzymes have an overall shape that resembles a ring with a central pore that might be involved in substrate processing. In the hexameric configuration, the ATP-binding site is positioned at the interface between the subunits. Upon ATP binding and hydrolysis, AAA enzymes undergo conformational changes in the AAA-domains as well as in the N-domains. These motions can be transmitted to substrate protein. AAA proteins are not restricted to eukaryotes. Prokaryotes have AAA which combine chaperone with proteolytic activity, for example in CIpAPS complex, which mediates protein degradation and recognition in E. coli. The basic recognition of proteins by AAAs is thought to occur through unfolded domains in the substrate protein. In HsIU, a bacterial CIpX/CIpY homologue of the HSP100 family of AAA+ proteins, the N- and C-terminal subdomains move towards each other when nucleotides are bound and hydrolysed. The terminal domains are most distant in the nucleotide-free state and closest in the ADP-bound state. Thereby the opening of the central cavity is affected. The AAA-type ATPase Cdc48p/p97 is perhaps the best-studied AAA protein. Misfolded secretory proteins are exported from the endoplasmic reticulum (ER) and degraded by the ER-associated degradation pathway (ERAD). Nonfunctional membrane and luminal proteins are extracted from the ER and degraded in the cytosol by proteasomes. Substrate retrotranslocation and extraction is assisted by the Cdc48p(Ufd1p/NpI4p) complex on the cytosolic side of the membrane. On the cytosolic side, the substrate is ubiquitinated by ER-based E2 and E3 enzymes before degradation by the 26S proteasome.

“Endoplasmic Reticulum Associated Protein Degradation” (ERAD) designates a cellular pathway which targets misfolded proteins of the endoplasmic reticulum for ubiquitination and subsequent degradation by a protein-degrading complex, called the proteasome. The process of ERAD can be divided into three steps. 1) The recognition of misfolded or mutated proteins in the endoplasmic reticulum depends on the detection of substructures within proteins such as exposed hydrophobic regions, unpaired cysteine residues and immature glycans. In mammalian cells for example, there exists a mechanism called glycan processing. In this mechanism, the lectin-type chaperones calnexin/calreticulin (CNX/CRT) provide immature glycoproteins the opportunity to reach their native conformation. They can do this by way of reglucosylating these glycoproteins by an enzyme called UDP-glucose-glycoprotein glucosyltransferase. Terminally misfolded proteins, however, must be extracted from CNX/CRT. This is carried out by EDEM and ER mannosidase I. This mannosidase removes one mannose residue from the glycoprotein and the latter is recognized by EDEM. Eventually EDEM will target the misfolded glycoproteins for degradation. 2) Because the ubiquitin-proteasome system (UPS) is located in the cytosol, terminally misfolded proteins have to be transported from the endoplasmic reticulum back into cytoplasm (Retro-translocation into the cytosol). A protein complex, called Sec61, constitutes the channel necessary for the transport of these misfolded proteins. Further, this translocation requires a driving force that determines the direction of transport. Since polyubiquitination is essential for the export of substrates, it is thought that this driving force is provided by ubiquitin-binding factors. One of these ubiquitin-binding factors is the Cdc48p-NpI4p-Ufd1p complex. It is known that human have the homolog of Cdc48p which is called Valosine-containing protein (VCP)/p97 and VCP/p97 have the same function of its yeast homolog Cdc48p. VCP/p97 transports substrates from endoplasmic reticulum to cytoplasm with its ATPase activity. 3) The ubiquitination of terminally misfolded proteins is caused by a cascade of enzymatic reactions (Ubiquitin-dependent degradation by the proteasome). The first of these reactions takes place when the ubiquitin-activating enzyme E1 hydrolyses ATP and forms a high-energy thioester linkage between a cysteine residue in its active site and the C-terminus of ubiquitin. The resulting activated ubiquitin is then passed to E2, which is an ubiquitin-conjugating enzyme. Another group of enzymes, more specifically ubiquitin protein ligases called E3, bind to the misfolded protein. Next they align the protein and E2, thus facilitating the attachment of ubiquitin to lysine residues of the misfolded protein.

Following successive addition of ubiquitin molecules to lysine residues of the previously attached ubiquitin, a polyubiquitin chain is formed. A polyubiquitinated protein is produced and this is recognized by specific subunits in the 19S capping complexes of the 26S proteasome. Hereafter, the polypeptide chain is fed into the central chamber of the 20S core region that contains the proteolytically active sites. Ubiquitin is cleaved before terminal digestion by deubiquitinating enzymes.

This third step is very closely associated with the second one, since ubiquitination takes place during the translocation event. However, the proteasomal degradation takes place in the cytoplasm. The ER membrane anchored RING finger containing ubiquitin ligases Hrd1 and Doa10 are the major mediators of substrate ubiquitination during ERAD. The tail anchored membrane protein Ubc6 as well as Ubc1 and the Cue1 dependent membrane bound Ubc7 are the ubiquitin conjugating enzymes involved in ERAD. As the variation of ERAD-substrates is enormous, several variations of the ERAD mechanism have been proposed. Indeed, it was confirmed that soluble, membrane and transmembrane proteins were recognized by different mechanisms. This led to the identification of 3 different pathways that constitute in fact 3 checkpoints. The first checkpoint is called ERAD-C and monitors the folding state of the cytosolic domains of membrane proteins. If defaults are detected in the cytosolic domains, this checkpoint will remove the misfolded protein. When the cytosolic domains are found to be correctly folded, the membrane protein will pass to a second checkpoint where the luminal domains are monitored. This second checkpoint is called the ERAD-L pathway. Not only membrane proteins surviving the first checkpoint are controlled for their luminal domains, also soluble proteins are inspected by this pathway as they are entirely luminal and thus bypass the first checkpoint. If a lesion in the luminal domains is detected, the involved protein is processed for ERAD using a set of factors including the vesicular trafficking machinery that transports misfolded proteins from the endoplasmic reticulum to the Golgi apparatus. Also a third checkpoint, called the ERAD-M pathway, has been described that relies on the inspection of transmembrane domains of proteins. “Valosin containing protein” (VCP; EntrezGenla 7415), also known as p97, MGC131997, Valosin-containing protein, MGC148092, IBMPFD, MGC8560, TERA, 15S Mg(2+)-ATPase p97 subunit, transitional endoplasmic reticulum ATPase, TER ATPase, or yeast Cdc48p homolog, is a member of a family that includes putative ATP-binding proteins involved in vesicle transport and fusion, 26S proteasome function, and assembly of peroxisomes. This protein, as a structural protein, is associated with clathrin, and heat-shock protein Hsc70, to form a complex. It has been implicated in a number of cellular events that are regulated during mitosis, including homotypic membrane fusion, spindle pole body function, and ubiquitin-dependent protein degradation. VCP is necessary for the fragmentation of Golgi stacks during mitosis and for their reassembly after mitosis. VCP is also involved in the formation of the transitional endoplasmic reticulum (tER). The transfer of membranes from the endoplasmic reticulum to the Golgi apparatus occurs via 50-70 nm transition vesicles which derive from part-rough, part-smooth transitional elements of the endoplasmic reticulum (tER). Vesicle budding from the tER is an ATP-dependent process. The ternary complex containing UFD1L, VCP and NPLOC4 binds ubiquitinated proteins and is necessary for the export of misfolded proteins from the ER to the cytoplasm, where they are degraded by the proteasome. The NPLOC4-UFD1L-VCP complex regulates spindle disassembly at the end of mitosis and is necessary for the formation of a closed nuclear envelope. VCP forms a homohexamer, which forms a ring-shaped particle of 12.5 nm diameter, that displays 6-fold radial symmetry. It is part of a ternary complex containing STX5A, NSFL1C and VCP. NSFL1C forms a homotrimer that binds to one end of a VCP homohexamer. The complex binds to membranes enriched in phosphatidylethanolamine-containing lipids and promotes Golgi membrane fusion. It can bind to a heterodimer of NPLOC4 and UFD1L, wherein the binding to this heterodimer inhibits Golgi-membrane fusion. Interaction with VCIP135 leads to dissociation of the complex via ATP hydrolysis by VCP. It is part of a ternary complex containing NPLOC4, UFD1L and VCP. VCP is a component of a complex required to couple retrotranslocation, ubiquitination and deglycosylation composed of NGLY1, SAKS1, AMFR, VCP and RAD23B.

Because the ubiquitin-proteasome system (UPS) is located in the cytosol, terminally misfolded proteins have to be transported from the endoplasmic reticulum back into cytoplasm by the “endoplasmic reticulum protein retrotranslocation machinery”. A protein complex, called Sec61, constitutes the channel necessary for the transport of these misfolded proteins. Further, this translocation requires a driving force that determines the direction of transport. This driving force is provided by ubiquitin-binding factors. In yeast, one of these ubiquitin-binding factors is the Cdc48p-NpI4p-Ufd1p complex. Humans have the homolog of Cdc48p which is called Valosine containing protein (VCP). VCP have the same function of its yeast homolog Cdc48p. VCP transports substrates from endoplasmic reticulum to cytoplasm with its ATPase activity.

Examples of inhibitors of the endoplasmic reticulum protein retrotranslocation machinery are p97 inhibitors. Examples of p97 inhibitors are:

“Eeyarestatin-I” (Eer-I; Chemical Name: 3-(4-Chlorophenyl)-4-[[[(4-chlorophenyl)amino]carbonyl]hydroxyamino]-5,5-dimethyl-2-oxo-1-imidazolidineacetic acid 2-[3-(5-nitro-2-furanyl)-2-propen-1-ylidene]hydrazide), this compound interferes with endoplasmic-reticulum associated degradation (ERAD) process by binding VCP and preventing the interaction with critical, yet unidentified, adaptor proteins. Eer-I has been shown to prevent the degradation of target misfolded proteins whose translocation and disposal is dependent upon VCP. (Wang Q et al. PLoS One. 2010 Nov. 12; 5(11):e15479. Wang Q et al. J Biol Chem. 2008 Mar. 21; 283(12):7445-54).

“DBEQ” (Chemical name: N²,N⁴-Dibenzylquinazoline-2,4-diamine) is a reversible, ATP-competitive direct VCP inhibitor identified for its specificity in preventing VCP-dependent proteasome degradation of target proteins. (Chou T F et al. Proc Natl Acad Sci USA. 2011 Mar. 2; 108(12):4834-9.

“Syk-inhibitor III” (Chemical name: 3,4-Methylenedioxy-β-nitrostyrene) is a tyrosine kinase inhibitor that was found to have a substantial efficacy as an irreversible inhibitor of the D2 ATPase site on VCP. (Chou T F et al. J Biol Chem. 2011 May 13; 286(19):16546-54)

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

Climb test: Neurodegenerative status of the mice was tested using a climb test device developed by some of the inventors. This device is based on a flappable wire meshwork platform (10×10 cm) suspended from one corner by a rigid boom. The platform is suspended about 40 cm above a surface. The platform is fully open and the test is performed in ambient light. During the test, mice are prevented of climbing the boom by a transparent plastic disk. For the performance of the test, mice were gently transferred to the platform and allowed to explore it for 2 minutes. The platform was flipped at the beginning of the test, and the mice (actively hanging to the grid with four limbs) spontaneously displayed the drive to reach the edge of the grid and climb up. As soon as the mice reached the upper surface of the grid (all four limbs on the same side), the grid was gently flipped over so that the mice were not be allowed to rest. The number of performed climbs in the test period was counted, the test period being 11 minutes (5 min and 30 min variants have been tested as well). The climb movement to be performed by the mice involves a forceful yet brief push with fore- and hind-limbs, thus mimicking short-lived but powerful movements that are known to recruit fast motoneurons.

Climb test detects early, gene-dosage dependent changes in the motor performance of mSOD1 trangenic mice: The climb test was applied to wild-type (wt) mice or to transgenic mice bearing either low-copies number (SSOD) or high-copies number (FSOD) of the SOD(G93A) transgene, which displays a progression rate proportional to the copies of the transgene. Wild type mice performed at least 40 climbs in the juvenile stage and thereafter stabilized their performance between 30 and 40 climbs per 11 minutes. SSOD mice displayed a performance comparable to the WT up to the age of 35 days (P35), then declining slowly toward performance scores about one-third of the WT mice. The performance of FSOD mice was impaired at the earliest time point tested. At the age of 30 days, FSOD mice performed at very low levels (performing on average 5 climbs vs. 30-35 of the wt), that were stable until late stages of disease.

Thus, the test is suitable to identify changes in motor performance at the very early stages of disease, i.e. before any typical disease phenotype. Moreover, the performance of the SOD mice in the climb test correlates with the amount of mutant protein expressed.

Eer-I delays motor disability in FSOD mice: Mice expressing mutant SOD1(G93A) were treated with EEr-I (1 μg/g in 150 mM NaCl, s.c., daily) from weaning (P20) or from P63. Motor performance was assessed by grid test, a standard task for the quantification of muscle strength and neurological impairment. Untreated FSOD mice displayed reduced strength at the earliest time point tested followed by progressive worsening. FSOD mice treated with Eer-I displayed a better performance at the earliest time point and a significantly slower decline in muscle strength over time. FSOD mice treated from day 63 onwards displayed a normal progression up to the start of the treatment, and displayed a fast improvement in performance soon after the beginning of said treatment. Although not reaching the same performance as the mice treated since weaning, rescued mice performed significantly better than their untreated counterparts.

Eer-I markedly improve FSOD mice performance in the climb test: Mice expressing mutant SOD1(G93A) were treated with EEr-I (1 μ/g in 150 mM NaCl, s.c., daily) from weaning (P20) or from P63. In the climb test the performance of FSOD mice was greatly reduced compared to wild-type already at the age of 28 days (P28), and quickly decreased to a score lower than 10 climbs/11 min. FSOD mice treated from P20 displayed a markedly preserved performance, with climb scores comparable with WT mice. The performance in the climb test remained stable up to day P65, when a slow, progressive decline began. FSOD mice treated at P63 displayed a marked but transient improvement in performance, with the climb score increasing up to four-fold but returning in the range of untreated FSOD mice at day P85.

Eer-1 delays body weight loss: Mice expressing mutant SOD1(G93A) were treated with EEr-I (1 μg/g in 150 mM NaCl, s.c., daily) from weaning (P20) or from P63. Body weight was recorded at P45, P65, P85 and then at 5-7 days intervals. Five mice per group were followed, representative individuals are shown. Whereas wild type mice treated with Eer-1 displayed the expected progressive increase in body weight (underscoring the lack of overt toxicity of the treatment), FSOD mice began losing weight progressively from P90 onwards, reaching at death a bodyweight loss of more than 40% as compared to wild-type mice. Eer-I treated FSOD mice displayed a stable bodyweight until day P140-145 when they entered a phase of rapid weight loss. In FSOD mice treated from P63, body weight stabilized at a level lower than the littermates treated from weaning, but the weight stabilized until the very latest stages of disease progression.

Eer-I prolongs FSOD mice survival: Mutant SOD1(G93A) transgenic mice (n=7 for each group) were treated since weaning (P20) with Eer-I (1 μg/g in 150 mM NaCl, s.c., daily) or saline. Survival was determined up to the day of death or euthanasia (according to the termination criteria of a righting time >30 sec). Treated mutant mice survived significantly longer than untreated mutant mice, with the longest-surviving treated mutant mouse living up to P176 (for untreated mutant mice, the longest survival observed was P135).

Eer-I treatment quickly restores performance of FSOD mice in the climb test: Two groups of FSOD mice were tested in the climb test at day P25 and P27, displaying a performance markedly lower than the wt. At day P27, mice were treated either with Eer-I (1 μg/g in 150 mM NaCl, s.c., daily) or with saline. Mice were tested at P29 and at P32. Whereas saline-treated FSOD mice progressively worsened in the climb task, mice treated with Eer-I displayed a marked improvement in performance, reaching after 5 days of treatment a level comparable with wt.

Eer-I effect is reversible upon withdrawal: FSOD mice (n=4) were treated with Eer-I (1 μg/g in 150 mM NaCl, s.c., daily) from P18 onwards and tested at P27, P37 and P47 displaying a climb performance comparable to wt mice. On day P47 treatment was stopped and the mice were tested 2 days later. At P49, i.e. after 2 days of withdrawal of treatment, all four mice experienced a sharp drop in performance, down to a level comparable with that of untreated FSOD mice of the same age. Upon resumption of the treatment, the mice quickly improved and after 2-5 days of treatment their performance was comparable with the performance before the withdrawal of the treatment.

The ATP-site competitive VCP inhibitor DBEQ improves motor performance in FSOD mice: Three FSOD mice were tested on the climb test on day 29 (i.e. a time point when they performed significantly worse than the wt) and thereafter treated with the VCP inhibitor DBEQ (1 μg/g in 150 mM NaCl, s.c., daily). Mice were tested repeatedly at day P31, P34, 37 and 45 and displayed a progressive improvement in motor performance reaching the range of wt mice.

The syk-inhibitor III (known to also inhibit VCP) improves motor performance in FSOD mice: Two mice were treated with the compound known as syk-inhibitor III, that was recently shown to be also an irreversible inhibitor of VCP. Treatment dose was 1 μg/g in 150 mM NaCl, s.c., daily. Mice were treated and tested at baseline (p32), and thereafter tested in the climb test at 38, 43, 56 days of age. The performance in the climb test markedly improved and reached the range of performance for WT mice.

Eer-I reverses VCP upregulation in FSOD mice: Mice were perfused with 4% PFA, spinal cord and kept overnight at 4° C. in PFA. After washing in PBS, lumbar (L2-L5) spinal cord was cryoprotected overnight in 30% sucrose. Lumbar spinal cord was embedded in OCT and sectioned at 50 μm. Sections were blocked in PBS-3% BSA −0.3% Triton X 100, and incubated double-overnight at 4 C in the same buffer plus anti VCP monoclonal antibody (Affinity Bioreagents) diluted 1:10,000 and polyclonal anti VAChT (Sigma) 1:1000. Sections were then briefly washed with PBS and incubated for 120 min at RT with goat-anti-rabbit (Alexa 647, Invitrogen) or goat-anti-mouse (Alexa 488, Invitrogen). Confocal images were acquired using an Zeiss LSM700 microscope, fitted with a 20×air objective. Images were processed using Imaris software. For the analysis of VCP labeling intensities, data were acquired with identical confocal settings, ensuring that signals at the brightest cells were not saturated, and that background levels outside clusters were still detectable. Images were then analyzed quantitatively using Image-Pro 5 software (Media Cybernetics). Signal intensities were acquired within areas inside cells, excluding areas lacking signal; background levels outside cells were subtracted from these values. Average cytoplasmic VCP staining intensity in motoneurons was significantly higher in FSOD+ motoneurons, compared to WT controls, but was significantly reduced (to an extent comparable with WT) in FSOD mice treated with Eer-I.

1-12. (canceled)

13. A method for treating a neurodegenerative disease in a subject comprising modulating the endoplasmic reticulum protein retrotranslocation machinery by administering to said subject a therapeutically effective amount of a modulator of said endoplasmic reticulum protein retrotranslocation machinery.

14. A method for treating a neurodegenerative disease in a subject comprising administering a therapeutically effective amount of a modulator of the AAA ATPase Valosin containing protein (VCP) to said subject.

15. The method of claim 13 wherein the modulator is a VCP inhibitor.

16. The method of claim 14 wherein the modulator is a VCP inhibitor.

17. The method of claim 15 wherein the VCP inhibitor is selected from the group consisting of Eeyarestatin-I, DBEQ and Syk-inhibitor III, and chemical derivatives thereof.

18. The method of claim 16 wherein the VCP inhibitor is selected from the group consisting of Eeyarestatin-I, DBEQ and Syk-inhibitor III, and chemical derivatives thereof.

19. The method of claim 13, wherein said neurodegenerative disease is Amyotrophic Lateral Sclerosis (ALS).

20. The method of claim 14, wherein said neurodegenerative disease is Amyotrophic Lateral Sclerosis (ALS).

21. The method of claim 13, wherein the modulator of the endoplasmic reticulum protein retrotranslocation machinery decreases or silences the expression of one or more component of the endoplasmic reticulum protein retrotranslocation machinery.

22. The method of claim 14, wherein the modulator of VCP decreases or silences the expression of one or more component of the endoplasmic reticulum protein retrotranslocation machinery.

23. The method of claim 21 wherein the component is the AAA ATPase Valosin containing protein (VCP).

24. The method of claim 22 wherein the component is the AAA ATPase Valosin containing protein (VCP).

25. The method of claim 21 wherein the modulator is a siRNA.

26. The method of claim 22 wherein the modulator is a siRNA.

27. The method of any of claim 13, wherein the modulator of the endoplasmic reticulum protein retrotranslocation machinery is an antibody specifically binding to a component of the endoplasmic reticulum protein retrotranslocation machinery.

28. The method of any of claim 14, wherein the modulator of the VCP is an antibody specifically binding to a component of the endoplasmic reticulum protein retrotranslocation machinery.

29. The method of claim 27 wherein the component is the AAA ATPase Valosin containing protein (VCP).

30. The method of claim 28 wherein the component is the AAA ATPase Valosin containing protein (VCP).

31. The method of claim 27 wherein the modulator is a monoclonal antibody.

32. The method of claim 28 wherein the modulator is a monoclonal antibody.

33. A method of diagnosing a neurodegenerative disease comprising the step of assessing the expression of the gene coding for the AAA ATPase Valosin containing protein (VCP), or the level of the product coded by said gene, wherein an increase in the level of expression of said gene or in the level of said gene product, as compared to the levels measured in a normal population, is indicative of a possible neurodegenerative disease.
